(19) United States
(12) Patent Application Publication
(10) Pub. No.: US 2025/0160367 A1
(43) Pub. Date: May 22, 2025

(54) ANTIVIRAL FEED COMPOSITION CONTAINING JAPANESE APRICOT JUICE AS ACTIVE INGREDIENT

(71) Applicants: In Hyeok BAEK, Tongyeong-si (KR); Geon Suk LEE, GEOJE-SI (KR)

(72) Inventors: In Hyeok BAEK, Tongyeong-si (KR); Geon Suk LEE, GEOJE-SI (KR)

(21) Appl. No.: 18/696,696

(22) PCT Filed: Jun. 8, 2022
(86) PCT No.: PCT/KR2022/008041

§ 371 Date: Dec. 27, 2024

(30) Foreign Application Priority Data
Jul. 12, 2021 (KR) 10-2021-0090909

(51) Int. Cl. A23K 20/195; A23K 10/30; A61K 36/736

(52) U.S. Cl. CPC A23K 20/195; A23K 10/30; A61K 36/736

(57) ABSTRACT

The present invention relates to an antiviral feed composition containing Japanese apricot juice as an active ingredient, in which the feed composition further contains Firmiana simplex extract, Robinia pseudoacacia extract, Petasites japonicus extract, Cinnamon verum extract, and Asparagus officinalis extract and has an excellent killing effect on viruses that cause various diseases in livestock and companion animals while being harmless to the human body, whereby the composition is identified to have excellent efficacy for maintaining health of livestock or companion animals or for controlling the growth thereof.

### TECHNICAL FIELD

The present invention relates to an antiviral feed composition containing Japanese apricot juice as an active ingredient.

### BACKGROUND ART

The ultimate goal of both public health and sanitation is the prevention of disease. Diseases are influenced by pathogens, hosts, and the environment. Among these factors, pathogens, which are an external factor, can be controlled by methods such as disinfection (Korean Veterinary Public Health Association, Veterinary Public Health Education Council, Veterinary Public Health, Moonundang, pp. 1-2, 1996). Humans have long used natural products to control pathogens or viruses.

Japanese apricot, which is the fruit of Prunus mume Sieb. et Zucc., has been used since ancient times to prevent and treat food poisoning, infectious diseases, etc. The use of Japanese apricot is still widely studied for food preservation, and its sterilizing effect is widely recognized because they contain citric and malic acids. Japanese apricot extracts are known to be effective against all Gram-negative and Gram-positive bacteria, including E. coli (O157:H77), Vibrio parahaemolyticus, and Salmonella typhimurium (according “Research on Antibacterial Activity and Active Substances of Prunus Plants (Seeds) of the Genus Prunus”, Master's thesis, Ewha Womans University, 1986, by Kyung Sook Kim and In Hwan Lee), and their inhibitory effects against viruses are still being studied. Influenza is a type of respiratory illness virus that is widespread in many types of animals, including humans as well as livestock, such as pigs, chickens, ducks, or horses. Coronaviruses (CCVs) are most often transmitted to children and young animals (Elsevier, 2008, 554; Advances in Virus Research, 2006, 66, 193-232). Meanwhile, CCVs often cause mild symptoms or no symptoms at all, but they can sometimes cause serious enteritis.

The inventors have conducted various studies on virus-related feed compositions using Japanese apricots, which have become issues in recent years, and found that feed compositions containing the sugar extract of Japanese apricots as an active ingredient had an inhibitory effect on various viruses, thereby such compositions can be used as feed compositions for livestock, such as cattle, pigs, and chickens, and pets, such as dogs and cats. On the basis of the found fact, the inventors have completed the present invention.

### DOCUMENTS OF RELATED ART

### Patent Document

Korean Patent Application Publication No. 10-2005-0117975 (Title of Invention: Natural Antioxidant Composition Containing Firmiana simplex Extract and Method of Preparing Same, Applicant: Jinsoo Kim, Publication date: Dec. 15, 2005).
Korean Patent No. 10-0528267 (Title of Invention: Virucidal Composition Using Natural Products, Applicant: R&L Life Science Co., Ltd., Registration date: Nov. 7, 2005)
Korean Patent No. 10-1633432 (Title of Invention: Use of Robinia pseudoacacia Extract for Amelioration and Treatment of Gamma Herpesvirus Infection, Applicant: R&L Life Science Co., Registration date: Jun. 20, 2016)

### DISCLOSURE

### Technical Problem

An objective of the present invention is to provide an antiviral feed composition containing Japanese apricot juice as an active ingredient.

### Technical Solution

The present invention relates to an antiviral feed composition containing Japanese apricot juice, Firmiana simplex extract, Robinia pseudoacacia extract, Petasites japonicus (also known as buttebur) extract, Cinnamon verum extract, and Asparagus officinalis L. extract.

The feed composition may contain 20 to 30 parts by weight of the Firmiana simplex extract, 5 to 14 parts by weight of the Robinia pseudoacacia extract, 20 to 30 parts by weight of the Petasites japonicus extract, 3 to 15 parts by weight of the Cinnamon verum extract, and 10 to 20 parts by weight of the Asparagus officinalis extract, based on 100 parts by weight of the Japanese apricot juice. The feed composition may be used as a feed additive for livestock or animals, and added to a compound feed in an amount of 10 to 2000 times the weight immediately before use of the compound feed.

The antiviral feed composition may contain a Japanese apricot sugar extract. The Japanese apricot sugar extract may be mixed in an amount of 10 to 30 parts by weight per 100 parts by weight of the Japanese apricot juice.

Each of the extracts may be obtained by preparing a raw material thereof and extracting the raw material with water, C1 to C4 alcohol, or a mixture thereof as a solvent. The solvent is preferably water.

In addition, the feed composition is characterized in that it has antibacterial efficacy.

Hereinafter, the present disclosure will be described in detail.

Japanese apricot juice used in the present disclosure is characterized in that it is obtained by crushing and pressing raw Japanese apricots to produce a liquid phase and (juicing) heating the liquid phase, and yellow Japanese apricots or blue Japanese apricots may be used. Alternatively, the liquid phase obtained by crushing, pressing, and juicing raw Japanese apricots may be first heated, centrifuged, filtered through a microfilter, and secondarily heated. The centrifugation is performed at 4° C. to 10° C. at 8000 to 10000 rpm for 5 to 30 minutes, preferably at 70° C. to 125° C. for 0.5 to 48 hours. Next, primary or secondary heating may be performed at 70° C. to 125° C. The microfilter that is used may have a pore size of 0.2 to 0.45 μm.

The composition of the present disclosure may further contain a Japanese apricot sugar extract. The Japanese apricot sugar extract used in the present disclosure may be a liquid phase that is osmotically extracted by leaving a mixture of sugar and Japanese apricots stationary for a certain period of time and then removing the dregs in the mixture. The Japanese apricot sugar extract may be a liquid phase that is osmotically extracted from a mixture of 50 to 200 parts by weight of the sugar per 100 parts by weight of the Japanese apricots by leaving the mixture stationary for 3 to 6 months. The extraction temperature for the Japanese apricot sugar extract may be in the range of from 4° C. to 37° C. and preferably in the range of from 15° C. to 30° C. The sugar may be any sweetener, and at least one selected from the group consisting of sugar, sugar powder, oligosaccharides, agave syrup, maple syrup, cactus sugar, and honey. When the amount of the sugar in the mixture is less than 50 parts by weight 100 parts by weight of the Japanese apricot, the mixture may not be fermented but be prone to spoilage. When more than 200 parts by weight of the sugar is mixed per 100 parts by weight of the Japanese apricot, the juice extracted from the mixture of the sugar and Japanese apricot may not be well mixed due to the excessive sugar content. When the extraction period is shorter than 3 months, the mixture is prone to spoilage. On the other hand, when the extraction period is longer than 6 months, acetic acid fermentation or alcohol fermentation may occur, and spoilage may also occur depending on the storage place or environment of the mixture.

In the present disclosure, the Firmiana simplex may include one or more elements selected from branches, fruits, and leaves thereof, and as the Robinia pseudoacacia, the woody part may be used.

In the feed composition, each of the extracts may be mixed with the Japanese apricot juice after lyophilization. However, in the case where the extract is obtained by using water as an extraction solvent, the extraction may be mixed directly with the undergoing lyophilization. Japanese apricot juice without Preferably, each of the extracts obtained by solvent extraction is prepared by adding 5 to 20 parts by weight of water relative to the raw sample, heating the resulting mixture at 70° C. to 90° C. for 1 to 10 hours, and removing the dregs in the mixture.

The Firmiana simplex extract, Robinia pseudoacacia extract, Petasites japonicus extract, Cinnamon verum extract, and Asparagus officinalis extract, which are the extracts utilized in the present disclosure, may be prepared by a method described below. Raw materials for the respective extracts may be prepared first, and the raw materials may be extracted with water, a C1 to C4 alcohol, or a mixed solution thereof as a solvent, in which the C1 to C4 alcohol may be selected from the group consisting of methanol, ethanol, propanol, isopropanol, butanol, and isobutanol.

The raw materials may be extracted under the following conditions: a temperature of 20° C. to 100° C. for a duration of 1 minute to 48 hours. The above processes may be repeated one to four times. As an extraction apparatus used to obtain the extracts, a widely used common extraction apparatus, an ultrasonic crushing extractor, or a fractionator may be used. The prepared extracts obtained through solvent extraction may be dried with hot air, dried under reduced pressure, or lyophilized remove the solvent. In addition, the extracts may be purified through column chromatography. The extracts may be used after undergoing fractionation or purification using one or more known methods that are used for separation and extraction of plant components, including extraction with an organic solvent (alcohol, ether, acetone, etc.), distribution of hexane and water, and column chromatography. The chromatography may be selected from the group consisting of silica gel column chromatography, LH-20 column chromatography, ion exchange resin chromatography, medium pressure liquid chromatography, thin layer chromatography, silica gel vacuum liquid chromatography, and high performance liquid chromatography.

The feed composition of the present disclosure may be used in its undiluted form or diluted form and may be added with a variety of excipients. That is, the composition may further contain an ingredient selected from the group consisting of water-soluble vitamins, oil-soluble vitamins, polymeric peptides, polymeric polysaccharides, sphingolipids, and seaweed extracts. Aside from the exemplified excipients, the composition may further contain an ingredient selected from the group consisting of oils, preservatives, bactericides, antioxidants, botanical extracts, pH adjusters, purified water, etc.

### Advantageous Effects

The present invention relates to an antiviral feed composition containing Japanese apricot juice as an active ingredient, in which the feed composition further contains Firmiana simplex extract, Robinia pseudoacacia extract, Petasites japonicus extract, Cinnamon verum extract, and Asparagus officinalis extract and has an excellent killing effect on viruses that cause various diseases in livestock and companion animals while being harmless to the human body, whereby the composition is identified to have excellent efficacy for maintaining health of livestock or companion animals or for controlling the growth thereof.

### BEST MODE

Hereinafter, preferred embodiments of the present disclosure will be described in detail. However, the present disclosure is not limited to the embodiments described herein and may be embodied in other forms. Rather, the embodiments are provided so that this disclosure will be thorough and complete and will fully convey the spirit of the invention to those skilled in the art.

### Examples 1 to 6. Preparation of Antiviral Feed Compositions

Green Japanese apricots were crushed and squeezed in their raw form, then then primarily heated at 90° C. for 1 hour. The heated liquid was centrifuged at 4° C. at 8000 rpm for 30 minutes, and filtered through a paper filter, and the filtrate was secondarily heated under the same conditions as in the primary heating. The secondary heated liquid was then filtered through a 0.2 μm filter to obtain Japanese apricot juice.

As the raw material of Firmiana simplex, a mixture of equal weights of branches, fruits, and leaves was used. As the raw material of Robinia pseudoacacia, a mixture of branches and the woody part of stems thereof was prepared. As the raw material of Cinnamon verum, the bark of Cinnamomum cassia was prepared. The Firmiana simplex, Robinia pseudoacacia, Cinnamon verum, and Asparagus officinalis were prepared as undried plants. 10 kg of water was added per 1 kg of each raw material, i.e., Firmiana simplex, Robinia pseudoacacia, Cinnamon verum, and Asparagus officinalis, followed by heating at 80° C. for 5 hours so as to be extracted, followed by removal of the dregs to obtain only liquid phase. Each liquid phase was cooled and then used as it is.

A mixture of equal weights of green Japanese apricots and sugar was left stationary at 25° C. or room temperature in a cool, shaded place for three months, and when enough juice was extracted from the Japanese apricots, the dregs were removed, and the liquid was collected to obtain Japanese apricot sugar extract.

Next, an antiviral feed composition was prepared by mixing ingredients as shown in Table 1 below based on 100 g of the Japanese Apricot juice.

TABLE 1

| | | | | | | | Japanese | |
|---|---|---|---|---|---|---|---|---|
| | Japanese | Firmiana | Robinia | Petasites | Cinnamon | Asparagus | apricot | |
| | apricot | simplex | pseudoacacia | japonicus | verum | officinalis | sugar | |
| | juice | extract | extract | extract | extract | extract | extract | Total |
| Conditions | (g) | (g) | (g) | (g) | (g) | (g) | (g) | (g) |
| Example 1 | 100 | 25 | 13 | 25 | 4 | 15 | 0 | 182 |
| Example 2 | 100 | 29 | 13 | 27 | 3 | 10 | 0 | 182 |
| Example 3 | 100 | 20 | 10 | 20 | 15 | 17 | 0 | 182 |
| Example 4 | 100 | 22 | 15 | 22 | 3 | 20 | 0 | 182 |
| Example 5 | 100 | 30 | 5 | 30 | 4 | 13 | 0 | 182 |
| Example 6 | 100 | 20 | 8 | 25 | 4 | 15 | 10 | 182 |

Some of the feed compositions formulated as in Table 1 were diluted 10 times by weight with purified water. Each of the diluted feed compositions was placed in a spray bottle and diluted again. Each of the diluted feed compositions was well mixed with a feed for pigs and calves, in which the amount of the feed was 100 times the weight of the diluted feed composition so that the concentration of the feed composition in the finally compound feed was 5% by weight.

### Comparative Examples 1 to 5. Preparation of Comparative Antiviral Feed Compositions

Comparative antiviral feed compositions were prepared under the conditions shown in Table 2. The ingredients and preparation method were the same as in Example 1.

TABLE 2

| | | | | | | | Japanese | |
|---|---|---|---|---|---|---|---|---|
| | Japanese | Firmiana | Robinia | Petasites | Cinnamon | Asparagus | apricot | |
| | apricot | simplex | pseudoacacia | japonicus | verum | officinalis | sugar | |
| | juice | extract | extract | extract | extract | extract | extract | Total |
| Conditions | (g) | (g) | (g) | (g) | (g) | (g) | (g) | (g) |
| Comparative | 182 | 0 | 0 | 0 | 0 | 0 | 0 | 182 |
| Example 1 | | | | | | | | |
| Comparative | 0 | 100 | 30 | 10 | 25 | 17 | 0 | 182 |
| Example 2 | | | | | | | | |
| Comparative | 100 | 30 | 32 | 0 | 20 | 0 | 0 | 182 |
| Example 3 | | | | | | | | |
| Comparative | 100 | 0 | 20 | 40 | 0 | 22 | 0 | 182 |
| Example 4 | | | | | | | | |
| Comparative | 0 | 100 | 32 | 0 | 0 | 50 | 0 | 182 |
| Example 5 | | | | | | | | |

### Experimental Example 1. Verification of Antiviral Efficacy

To confirm the antiviral activity in livestock or animals, the feed compositions of Examples 1 to 6 and Comparative Examples 1 to 5 were assayed for antiviral activity by infecting the Mardin Darby Canine Kidney (MDCK) cell line with avian influenza H9N1 and H9N2, and then the cell line was treated with each of the feed compositions, in which each of the feed compositions was first lyophilized, and then each of the lyophilized feed compositions was converted to an aqueous solution in which the feed composition was contained at a concentration of 2 to 10 mg/ml. The MDCK cell line used in the experiment was prepared by routine culture in Eagle's minimum essential medium (MEM) containing 10% fetal bovine serum (FBS, Hyclone Thermo Scientific) and 1% penicillin-streptomycin solution (Gibco).

1×10⁴ MDCK cells per ml were dispensed into 96-well plates and incubated at 37° C. for 16 hours in a 5% CO₂ incubator. The monolayer MDCK cells were washed twice with PBS, infected with H9N1 and H9N2 at 100 TCID₅₀ at 37° C. for 2 hours, and the remaining viruses were washed out.

The infected cells were incubated in MEM culture medium at 37° C. for 48 hours until the viral cytopathic effect (CPE) appeared, in which the MEM culture medium contained Trypsin-TPCK, which is trypsin treated with 1 μg/ml of L-1-tosylamido-2-phenylethyl chloromethyl ketone, and a viral culture solution (0.3% bovine serum albumin, 1% Penicillin-Streptomycin solution) containing one of the sequentially diluted compositions of the examples and comparative examples. The antiviral CPE inhibiting ability of each of the compositions was determined by the 50% effective concentration value (EC₅₀) for viral inhibition, and the 50% cytotoxic concentration value (CC₅₀) was determined on the basis of the morphologic transformation of the cells. The anti-influenza virus effect of each of the compositions was expressed as the selectivity index (SI), which is the CC₅₀ value divided by the EC₅₀ value. The selectivity index (SI) is CC₅₀/EC₅₀, and the higher the SI value, the greater the inhibitory effect on virus proliferation. Table 3 shows the results of comparing the anti-influenza virus activity of each of the compositions against H9N1 and H9N2 in MDCK cells.

TABLE 3

| | CC₅₀ (μg/ml) | | EC₅₀ (μg/ml) | | SI | |
|---|---|---|---|---|---|---|
| Conditions | H9N1 | H9N2 | H9N1 | H9N2 | H9N1 | H9N2 |
| Example 1 | 7523 | 7623 | 43 | 31 | 175.0 | 245.9 |
| Example 2 | 7569 | 7523 | 41 | 42 | 184.6 | 179.1 |
| Example 3 | 7345 | 7634 | 42 | 34 | 174.9 | 224.5 |
| Example 4 | 7105 | 7632 | 34 | 35 | 209.0 | 218.1 |
| Example 5 | 7352 | 7345 | 32 | 35 | 229.8 | 209.9 |
| Example 6 | 7562 | 7235 | 25 | 21 | 302.5 | 344.5 |
| Comparative | 7803 | 7234 | 134 | 321 | 58.2 | 22.5 |
| Example 1 | | | | | | |
| Comparative | 7852 | 7412 | 126 | 245 | 62.3 | 30.3 |
| Example 2 | | | | | | |
| Comparative | 7245 | 7356 | 212 | 234 | 34.2 | 31.4 |
| Example 3 | | | | | | |
| Comparative | 7523 | 7315 | 341 | 132 | 22.1 | 55.4 |
| Example 4 | | | | | | |
| Comparative | 7983 | 7744 | 231 | 125 | 34.6 | 62.0 |
| Example 5 | | | | | | |

As shown in Table 3, it can be seen that the feed compositions of Examples 1 to 6 have large SI values showing a anti-viral effect than the feed compositions of 10 better Comparative Examples 1 to 5.

### Experimental Example 2. Verification of Antiviral Efficacy

The feed compositions of Examples 1 to 7 of the present invention were diluted in 10 times by weight in purified water, and each of the diluted feed compositions were placed in a spray bottle and then diluted again. Each of the diluted feed compositions was well mixed with a feed for young pigs and calves with stomatitis due to herpesvirus, in which the amount of the feed was 100 times the weight of the added feed composition so that the concentration of the feed composition in the compound feed was 5% by weight. Afterwards, the weight gain rate was compared among the pigs or calves for one month.

TABLE 4

| | Weight gain rate | Weight gain rat of |
|---|---|---|
| Conditions | of pigs (%) | calves (%) |
| Untreated | 100.0% | 100.0% |
| Example 1 | 156.5 | 152.0 |
| Example 2 | 157.2 | 149.3 |
| Example 3 | 156.3 | 161.1 |
| Example 4 | 163.0 | 148.2 |
| Example 5 | 146.3 | 153.4 |
| Example 6 | 187.4 | 179.5 |
| Comparative | 134.3 | 125.6 |
| Example 1 | | |
| Comparative | 125.1 | 132.0 |
| Example 2 | | |
| Comparative | 136.5 | 135.2 |
| Example 3 | | |
| Comparative | 126.0 | 132.3 |
| Example 4 | | |
| Comparative | 134.4 | 123.1 |
| Example 5 | | |

The results show that, as shown in Table 4, each of the calves and pigs that were fed the compounded feed containing any one of the compositions of Examples 1 to 6 exhibited improved stomatitis wounds caused by herpesvirus, and exhibited increased feed intake and a significantly improved weight gain.

### Experimental Example 3. Verification of Antiviral Effect

The pathogenic bacterium Escherichia coli 0-157 was primarily incubated in 3% (w/v) trypticase soy broth (TSB) at 37° C., 200 rpm for 18 hours, and then secondarily incubated under the same conditions for 2.5 hours so that the cell concentration became 4×10⁶ colony forming units (CFU)/ml. The cultured cells (4×10⁶ colony forming units (CFU)/ml) were added to and mixed with 10 ml of a sterilized gel (underlay gel) composed of citrate phosphate buffer (9 mM sodium phosphate, 1 mM sodium citrate, pH 7.4), 1% (w/v) type I (low electroendosmosis) agarose, and 0.03% (w/v) TSB, and poured into a square plate. When the gel solidified, a 6 mm paper disc was placed in the center on the plate, and 100 μl of a 100-fold dilution of the composition of each of Examples 1 to 5 and Comparative Examples 1 to 5 was dropped. After 2 days of incubation, the formation of the clear zone was checked, and the size of the clear zone was measured from the circular end of the specimen in an outward direction and listed in Table 5 below.

TABLE 5

| Specimen | E. coli (mm) |
|---|---|
| Example 1 | 16 |
| Example 2 | 16 |
| Example 3 | 15 |
| Example 4 | 15 |
| Example 5 | 16 |
| Example 6 | 23 |
| Comparative | 3 |
| Example 1 | |
| Comparative | 2 |
| Example 2 | |
| Comparative | 4 |
| Example 3 | |
| Comparative | 5 |
| Example 4 | |
| Comparative | 2 |
| Example 5 | |

The measurement results show that the antiviral effect of Examples 1 to 6 is significantly better than that of Comparative Examples 1 to 5.

1. An antiviral feed composition comprising Japanese apricot juice as an active ingredient.

2. The antiviral feed composition of claim 1, wherein the composition comprises Firmiana simplex extract, Robinia pseudoacacia extract, Petasites japonicus, Cinnamon verum extract, and Asparagus officinalis extract.

3. The antiviral feed composition of claim 2, wherein the composition comprises Japanese apricot sugar extract.

4. The antiviral feed composition of claim 1, wherein the Japanese apricot juice is obtained by heating a liquid phase obtained by crushing and pressing raw Japanese apricots.

5. The antiviral feed composition of claim 1, wherein the composition has an antiviral effect.
